(19) United States
(12) Patent Application Publication
(10) Pub. No.: US 2014/0161221 A1
(43) Pub. Date: Jun. 12, 2014

(54) X-RAY COMPUTED TOMOGRAPHY APPARATUS

(71) Applicants: Toshiba Medical Systems Corporation, Otawara-shi (JP); Kabushiki Kaisha Toshiba, Minato-ku (JP)

(72) Inventors: Hiroaki Miyazaki, Otawara-shi (JP); Sanae Harada, Nasushiobara-shi (JP)

(73) Assignees: Toshiba Medical Systems Corporation, Otawara-shi (JP); Kabushiki Kaisha Toshiba, Minato-ku (JP)

(21) Appl. No.: 14/179,149

(22) Filed: Feb. 12, 2014

Related U.S. Application Data
(63) Continuation of application No. PCT/JP2013/073449, filed on Aug. 30, 2013.

(30) Foreign Application Priority Data
Aug. 30, 2012 (JP) 2012-190481
Aug. 23, 2013 (JP) 2013-173929

(51) Int. Cl. A61B 6/03; A61B 6/00

(52) U.S. Cl. CPC A61B 6/032; A61B 6/405
USPC 378/16

(57) ABSTRACT

An X-ray computed tomography apparatus according to an embodiment includes an X-ray tube, a bias power supply, and an X-ray control unit. The X-ray tube includes a cathode, an anode, and a grid between the cathode and the anode. The bias power supply generates a bias voltage to be applied to the grid to control a tube current between the cathode and the anode. The X-ray control unit applies a bias voltage as a pulse string for generating a constant tube current and controls at least one of the pulse count and pulse width of a bias voltage for each predetermined period.

### CROSS-REFERENCE TO RELATED APPLICATIONS

This application is a Continuation application of PCT Application No. PCT/JP2013/073449, filed Aug. 30, 2013 and based upon and claims the benefit of priority from the Japanese Patent Application No. 2012-190481, filed Aug. 30, 2012 and the Japanese Patent Application No. 2013-173929, filed Aug. 23, 2013, the entire contents of all of which are incorporated herein by reference.

### FIELD

Embodiments described herein relate generally to an X-ray computed tomography apparatus.

### BACKGROUND

In general, an X-ray computed tomography apparatus (to be referred to as an X-ray CT apparatus) detects the X-rays emitted from an X-ray tube to an object (the X-rays transmitted through the object) via an X-ray detector. The X-ray CT apparatus reconstructs an internal image of the object based on an output (projection data) from the X-ray detector.

An X-ray CT apparatus configured to perform tube voltage switching control and the like has recently been required to control an X-ray tube current at high speed from the viewpoints of the performance of the X-ray CT apparatus and a reduction in radiation exposure on an object.

With regard to a tube current control technique, there is known a triode X-ray tube having a triode structure including a cathode, an anode, and a grid (bias electrode).

When an X-ray CT apparatus uses such a triode X-ray tube, the X-ray CT apparatus controls a bias voltage on the grid (to be referred to as a grid voltage hereinafter) of the triode X-ray tube. This controls thermoelectron emission from the filament. Controlling thermoelectron emission will control an increase/decrease in X-ray tube current.

When using a triode X-ray tube for an X-ray CT apparatus and controlling an X-ray tube current with a grid voltage as described above, it is possible to decrease the X-ray tube current by applying a high grid voltage. In this case, however, the focus size of the X-ray CT apparatus decreases.

When an X-ray tube current is increased by decreasing a grid voltage, the focus size of the X-ray CT apparatus increases.

When, for example, the focus size of each projection data used for the reconstruction of the same image changes in accordance with grid voltage control, the resolution of each of the projection data changes.

Such a change in the resolution of each projection data used for the reconstruction of the same image (the difference in resolution between the respective projection data) will degrade image quality due to the uneven resolution of the image reconstructed from the projection data or will cause artifacts.

Note that controlling a current (filament current) supplied to the filament of the X-ray tube will control thermoelectron emission based on a change in the temperature of the filament, instead of controlling an X-ray tube current by using the above grid voltage. This makes it possible to control an X-ray tube current.

When controlling a filament current, however, since a change in filament temperature influences a change in thermoelectron, it is difficult to implement high-speed X-ray tube current control with a large time constant.

### BRIEF DESCRIPTION OF THE DRAWINGS

FIG. 1 is a block diagram showing the arrangement of an X-ray computed tomography apparatus according to an embodiment.

FIG. 2 is a block diagram for explaining the arrangement of the X-ray tube apparatus and X-ray control/high voltage generator shown in FIG. 1 according to the embodiment.

FIG. 3 is a graph for explaining control on an X-ray tube current by control on the pulse count of a grid voltage according to the embodiment.

FIG. 4 is a graph showing the correlation between grid voltage and X-ray tube current.

FIG. 5 is a graph showing the correlation between grid voltage and focus size.

FIG. 6 is a graph for explaining control on an X-ray tube current by control on the pulse width of a grid voltage according to the embodiment.

FIG. 7 is a view showing an example of the relationship between an object and the relative position of an X-ray tube according to a modification of the embodiment.

FIG. 8 is a graph for explaining control on the pulse count of a grid voltage and control on a filament current according to the modification of the embodiment.

FIG. 9 is a graph for explaining control on the pulse width of a grid voltage and control on a filament current according to the modification of the embodiment.

FIG. 10 is a graph for explaining the maximum and minimum values of a tube current according to the modification of the embodiment.

FIG. 11 is a graph showing an example of the control width of a tube current corresponding to a view angle according to the modification of the embodiment.

### DETAILED DESCRIPTION

An X-ray computed tomography apparatus according to an embodiment includes an X-ray tube, a bias power supply, and an X-ray control unit.

The X-ray tube includes a cathode, an anode, and a grid placed between the cathode and the anode.

The bias power supply generates a bias voltage applied to the grid to control a tube current between the cathode and the anode.

The X-ray control unit applies the bias voltage as a pulse string which generates the constant tube current and controls at least one of a pulse count and pulse width of the bias voltage for each predetermined period.

An X-ray computed tomography apparatus (to be referred to as an X-ray CT apparatus hereinafter) according to an embodiment will be described with reference to the accompanying drawings.

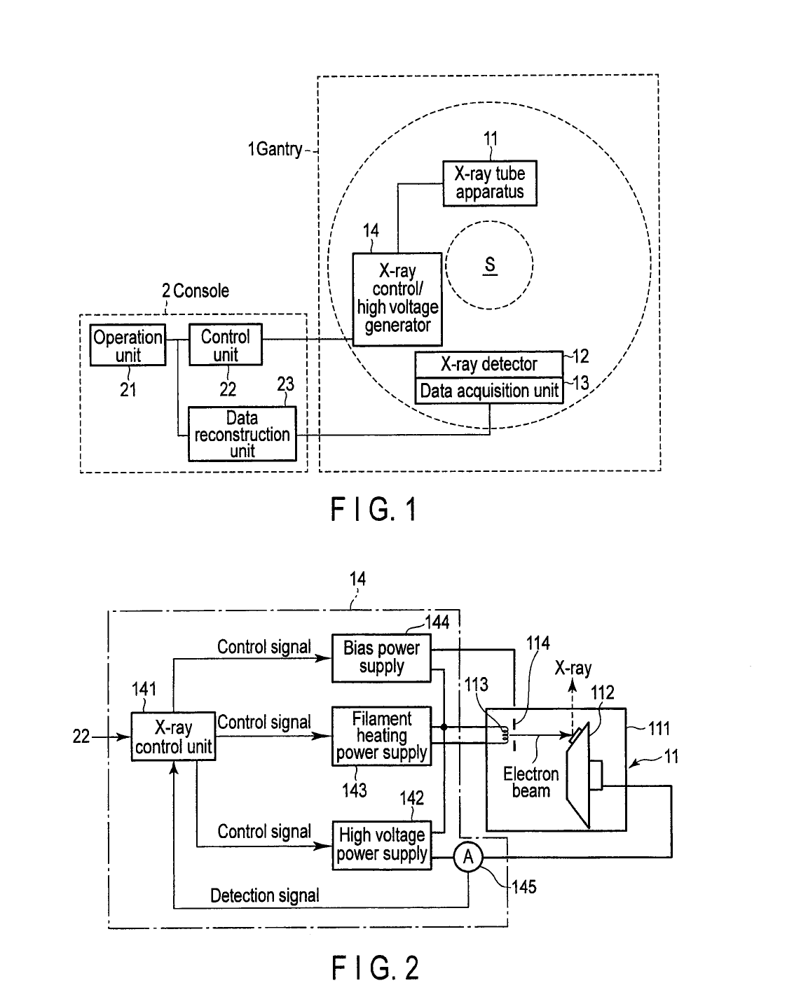

FIG. 1 shows the arrangement of the X-ray CT apparatus according to this embodiment. As shown in FIG. 1, the X-ray CT apparatus includes a gantry 1 configured to acquire projection data concerning an object and a console 2 which accommodates a plurality of modules necessary for control on the gantry 1 and various types of signal processing such as image reconstruction.

The gantry 1 includes an X-ray tube apparatus 11, an X-ray detector (multichannel X-ray detector) 12, a data acquisition unit 13, and an X-ray control/high voltage generator 14.

The X-ray tube apparatus 11 and the X-ray detector 12 are mounted on a ring-like frame which is rotated and driven. The X-ray tube apparatus 11 faces the X-ray detector 12 through an imaging area S in which an object is inserted at the time of imaging.

The X-ray tube apparatus 11 includes an X-ray tube which generates X-ray. Note that the X-ray tube of the X-ray CT apparatus according to this embodiment is a triode X-ray tube including a cathode, an anode, and a grid (bias electrode) placed between the cathode and the anode. Note that the arrangement of the X-ray tube apparatus 11 will be described in detail later.

The X-ray detector 12 detects the X-rays generated from the X-ray tube and transmitted through the object inserted in the imaging area S.

The data acquisition unit 13 acquires projection data concerning an object based on an output from the X-ray detector 12. More specifically, the data acquisition unit 13 converts the signal output from the X-ray detector 12 for each channel into a voltage signal, amplifies the signal, and outputs it upon converting it into a digital signal (projection data).

The X-ray control/high voltage generator 14 controls an X-ray tube current by controlling the bias voltage (to be referred to as a grid voltage hereinafter) applied to the above grid. Note that the detailed arrangement of the X-ray control/high voltage generator 14 will be described below.

The console 2 includes an operation unit 21 with which the operator inputs scan conditions and the like, a control unit 22 for executing a scan by controlling the overall apparatus in accordance with the scan conditions set by the operator, and a data reconstruction unit 23 which reconstructs image data concerning a slice or volume based on the projection data acquired by the data acquisition unit 13.

The arrangement of the X-ray tube apparatus 11 and X-ray control/high voltage generator 14 of the X-ray CT apparatus according to this embodiment will be described next with reference to FIG. 2.

As shown in FIG. 2, the X-ray tube apparatus 11 includes a triode X-ray tube 111 in a closed vacuum state. The triode X-ray tube 111 accommodates an anode (rotating anode) 112, a cathode 113 facing the anode 112, and a grid 114 placed between the anode 112 and the cathode 113. It is possible to control the emission and stop of X-rays from the triode X-ray tube 111 by using a grid voltage (the voltage applied to the grid 114).

The X-ray control/high voltage generator 14 includes an X-ray control unit 141, a high voltage power supply 142, a filament heating power supply 143, a bias power supply 144, and a tube current detection unit 145.

The X-ray control unit 141 controls the high voltage power supply 142, the filament heating power supply 143, and the bias power supply 144 in accordance with scan conditions and the like from the control unit 22 described above.

The high voltage power supply 142 generates a high voltage (tube voltage) to be applied between the anode 112 and the cathode 113 in accordance with a control signal from the X-ray control unit 141. The X-ray detector 142 applies (outputs) a tube voltage between the anode 112 and the cathode 113 in accordance with a control signal from the X-ray control unit 141. The high voltage power supply 142 stops generation or application of a tube voltage in accordance with a control signal from the X-ray control unit 141.

The filament heating power supply 143 generates a current (filament current) to be supplied to the filament of the cathode 113 in accordance with a control signal from the X-ray control unit 141. The filament heating power supply 143 supplies (outputs) a filament current to the filament of the cathode 113 in accordance with a control signal from the X-ray control unit 141. The filament heating power supply 143 stops generation or supply of a filament current in accordance with a control signal from the X-ray control unit 141.

The bias power supply 144 is an inverter type power supply which generates a grid voltage. The bias power supply 144 generates a grid voltage as a pulse sequence by using a switching element synchronous with a control pulse from the X-ray control unit 141. Note that the X-ray control unit 141 controls the bias power supply 144 so as to control the pulse count of a grid voltage for generating a constant tube current for each predetermined period (e.g., a data acquisition period).

The potential of the grid 114 changes between 0 and a potential with negative polarity (cutoff voltage) equal to or lower than the potential of the cathode 113.

Note that when the potential of the grid 114 is 0, the thermoelectrons emitted from the filament of the cathode 113 pass through the grid 114 and collide with a target such as tungsten of the rotating anode 112. As a result, a tube current flows. In contrast, when the potential of the grid 114 is at the cutoff potential, the grid 114 cuts off the thermoelectrons emitted from the filament of the cathode 113. Therefore, no tube current flows.

The tube current detection unit 145 detects an X-ray tube current. For example, the X-ray tube current detected by the tube current detection unit 145 is used for control on an X-ray tube current by the X-ray control unit 141.

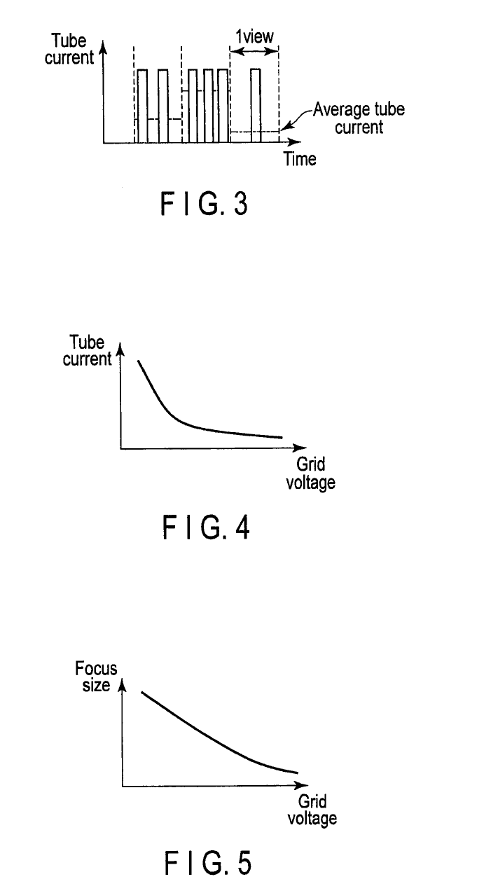

Control on an X-ray tube current in the X-ray. CT apparatus according to this embodiment will be described in detail below with reference to FIG. 3. Note that the X-ray control/high voltage generator 14 (the X-ray control unit 141 of the X-ray control/high voltage generator 14) described above executes control on an X-ray tube current.

In this embodiment, as shown in FIG. 3, a tube voltage is continuously applied between the anode 112 and the cathode 113 during a scan period in the X-ray CT apparatus. Furthermore, a filament current is continuously supplied to the filament. In addition, the apparatus applies a grid voltage for generating a constant tube current, as a pulse string, to the grid 114 and controls the pulse count of the grid voltage for each predetermined period.

More specifically, the apparatus controls the pulse count of a grid voltage for generating a constant tube current in synchronism with a data acquisition period of the data acquisition unit 13, for example, a minimum data acquisition period (one view), for each data acquisition period.

This allows the X-ray CT apparatus according to this embodiment to control an X-ray tube current by controlling an average tube current (value) during one view.

When, for example, the apparatus continuously changes a grid voltage, an increase in grid voltage will decrease a tube current, and vice versa, as shown in FIG. 4. As described above, it is also possible to control an X-ray tube current by continuously changing a grid voltage.

When, however, the apparatus continuously changes a grid voltage, for example, an increase in grid voltage for a decrease in tube current will decrease a focus size, as shown in FIG. 5. In contrast, decreasing a grid voltage to increase a tube current will increase a focus size. Reconstructing the same image from projection data having undergone changes in focus size in this manner will cause a deterioration in image quality or artifacts.

In contrast to this, as shown in FIG. 3, when the apparatus changes the pulse count of a grid voltage for generating a constant tube current in synchronism with the shortest data acquisition period, it is possible to perform control to increase or decrease an average tube current during the minimum period. In addition, since the wave height (tube current value) of a unit pulse does not change, the focus size does not change.

As described above, this embodiment is configured to continuously apply a tube voltage to continuously supply a filament current, apply a grid voltage (bias voltage), as a pulse string, for generating a constant tube current, and control the pulse count of the grid voltage for each predetermined period. This arrangement can control an X-ray tube current without changing a focus size because the wave height of the unit pulse does not change. In addition, using a grid voltage makes it possible to perform high-speed, fine control (adjustment) on a tube current in a wide range (wide variable width) as compared with tube current control using a change in temperature based on control on a filament current.

In addition, since this embodiment is configured to control the pulse count of a grid voltage in synchronism with a data acquisition period (one view) for each data acquisition period, it is possible to control an X-ray tube current (average tube current) more accurately for each data acquisition period.

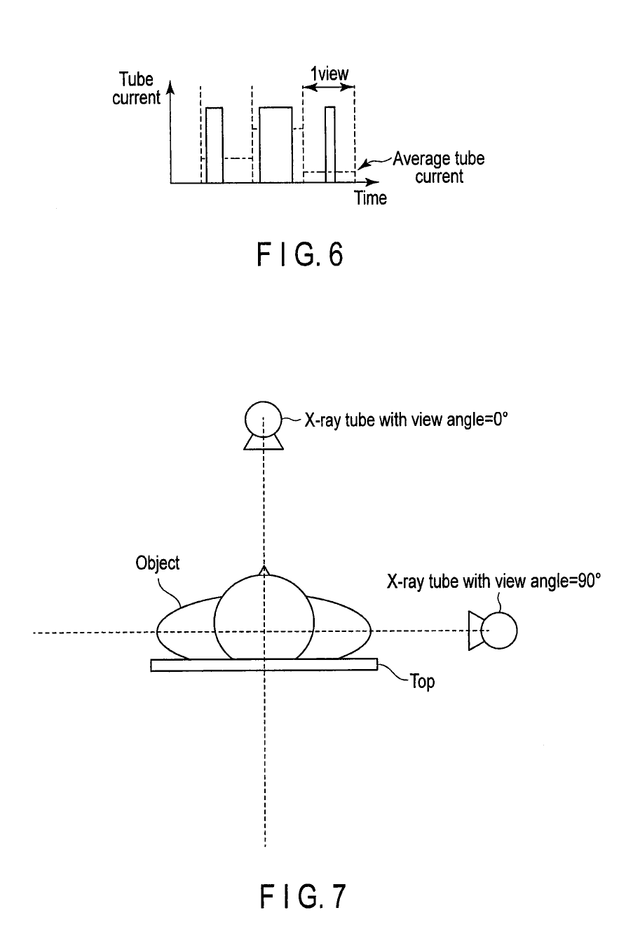

This embodiment has exemplified the apparatus configured to control the pulse count of a grid voltage for generating a constant tube current. For example, however, the apparatus may be configured to control an average tube current by controlling the pulse width of a grid voltage for generating a constant tube current in synchronism with the minimum data acquisition period (one view), as shown in FIG. 6. In this case as well, since the wave height of a unit pulse does not change, it is possible to control an X-ray tube current at high speed without changing a focus size.

Note that the X-ray control unit 141 can also control the bias power supply 144 to control the pulse width and pulse count of a grid voltage. This can more finely change an average tube current.

Note that X-ray CT apparatuses include various types of apparatuses, e.g., a rotate/rotate-type apparatus in which an X-ray tube and a radiation detector rotate together around an object, and a stationary/rotate-type apparatus in which many detection elements are arrayed in the form of a ring, and only an X-ray tube rotates around an object. This embodiment can be applied to either type.

In order to reconstruct tomographic image data corresponding to one slice, projection data corresponding to one rotation around an object, i.e., about 360°, is required, or 180°+α (α: fan angle) projection data is required in the half scan method. This embodiment can be applied to either of these reconstruction schemes.

As mechanisms of converting incident X-rays into electric charges, the following techniques are the mainstream: an indirect conversion system that converts X-rays into light through a phosphor such as a scintillator and converts the converted light into electric charges through photoelectric conversion elements such as photodiodes, and a direct conversion system that uses generation of electron-hole pairs in a semiconductor by X-rays and migration of the electron-hole pairs to an electrode, i.e., a photoconductive phenomenon. As an X-ray detection element, either of these schemes can be used.

Recently, with advances toward the commercialization of a so-called multi-tube type X-ray CT apparatus having a plurality of pairs of X-ray tubes and X-ray detectors mounted on a rotating ring, related techniques have been developed. This embodiment can be applied to both a conventional single-tube type X-ray CT apparatus and a multi-tube type X-ray CT apparatus.

### (Modification)

This modification differs from the above embodiment in changing a tube current in accordance with the position of the X-ray tube relative to an object by adjusting a filament current.

FIG. 7 shows an example of the relationship between an object and the relative position of the triode X-ray tube 111. As shown in FIG. 7, the apparatus changes a tube current by changing the magnitude of a filament current in accordance with the position of the triode X-ray tube 111 relative to the object, i.e., a view angle (view direction).

The X-ray control unit 141 controls the filament heating power supply 143 to change the magnitude of a tube current in accordance with the position of the triode X-ray tube 111 relative to the object. More specifically, the X-ray control unit 141 controls the filament heating power supply 143 to change the magnitude of a tube current in accordance with a view angle indicating the position of the X-ray tube. That is, the X-ray control unit 141 controls the filament heating power supply 143 to change a filament current in accordance with a view angle while controlling the bias power supply 144.

When, for example, the X-ray tube 111 is located at a view angle of 0° or 180°, i.e., the X-ray tube 111 is located in a direction perpendicular to the top on which the object is placed, during a scan on the object, the X-ray control unit 141 controls the filament heating power supply 143 so as to decrease a tube current. At this time, the X-ray control unit 141 controls the filament heating power supply 143 so as to decrease a filament current. In addition, when the X-ray tube 111 is located at a view angle of 90° or 270°, i.e., the X-ray tube 111 is located in the short-axis direction of the top on which the object is placed, during a scan on the object, the X-ray control unit 141 controls the filament heating power supply 143 so as to increase a tube current. At this time, the X-ray control unit 141 controls the filament heating power supply 143 so as to increase a filament current.

That is, when the X-ray tube 111 is located at a view angle of 0° or 180°, the X-ray control unit 141 controls the filament heating power supply 143 so as to minimize a filament current during a scan. When the X-ray tube 111 is located at a view angle of 90° or 270°, the X-ray control unit 141 controls the filament heating power supply 143 so as to minimize a filament current during a scan. In other words, the X-ray control unit 141 controls the filament heating power supply 143 such that a filament current when the triode X-ray tube 111 is located in a direction perpendicular to the top on which the object is placed becomes smaller than a filament current when the X-ray tube 111 is located in the short-axis direction of the top. With this operation, a tube current when the X-ray tube 111 is located in the direction perpendicular to the top on which the object is placed becomes smaller than a tube current when the triode X-ray tube 111 is located in the short-axis direction of the top. That is, the X-ray control unit 141 controls the filament heating power supply 143 to indirectly control a tube current.

Note that the X-ray control unit 141 may decide a filament current corresponding to a view angle in accordance with the body thickness of the object which is set in advance via an input unit (not shown). Alternatively, the X-ray control unit 141 may decide the body thickness of the object in a pre-scan executed for the object before an actual scan and decide a filament current based on the decided body thickness of the object.

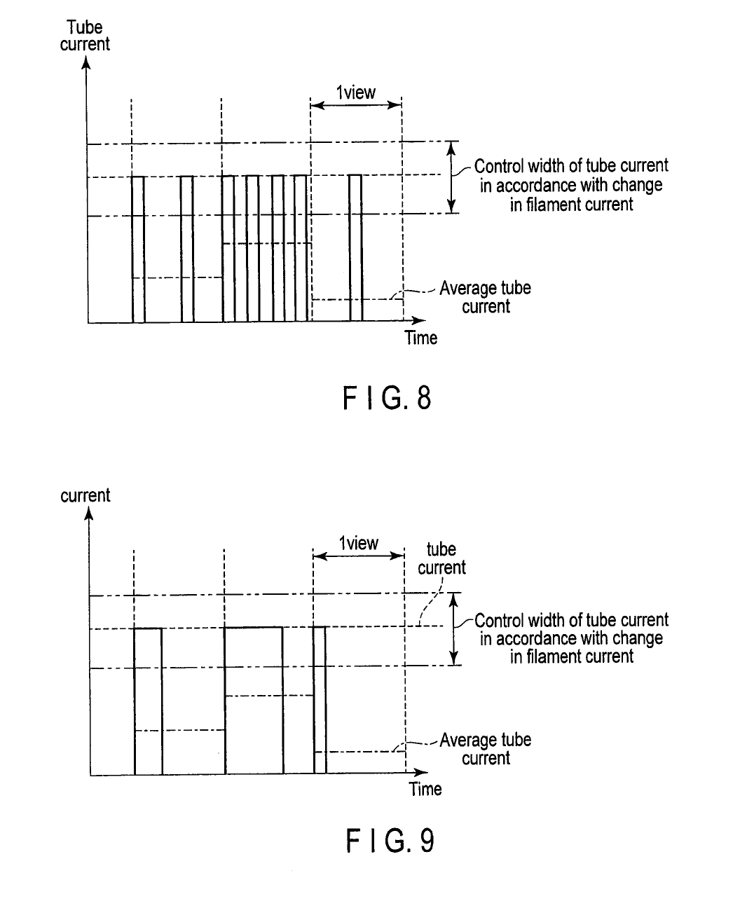

FIG. 8 is a graph for explaining control on the pulse count of a grid voltage and control on a filament current in this modification. As shown in FIG. 8, adding control on a filament current to control the pulse count of grid voltage can change an X-ray tube current in accordance with a view angle without changing a focus size.

FIG. 9 is a graph for explaining control on the pulse width of a grid voltage and control on a filament current. As shown FIG. 8, adding control on a filament current to control on the pulse width of a grid voltage can change an X-ray tube current in accordance with a view angle without changing a focus size.

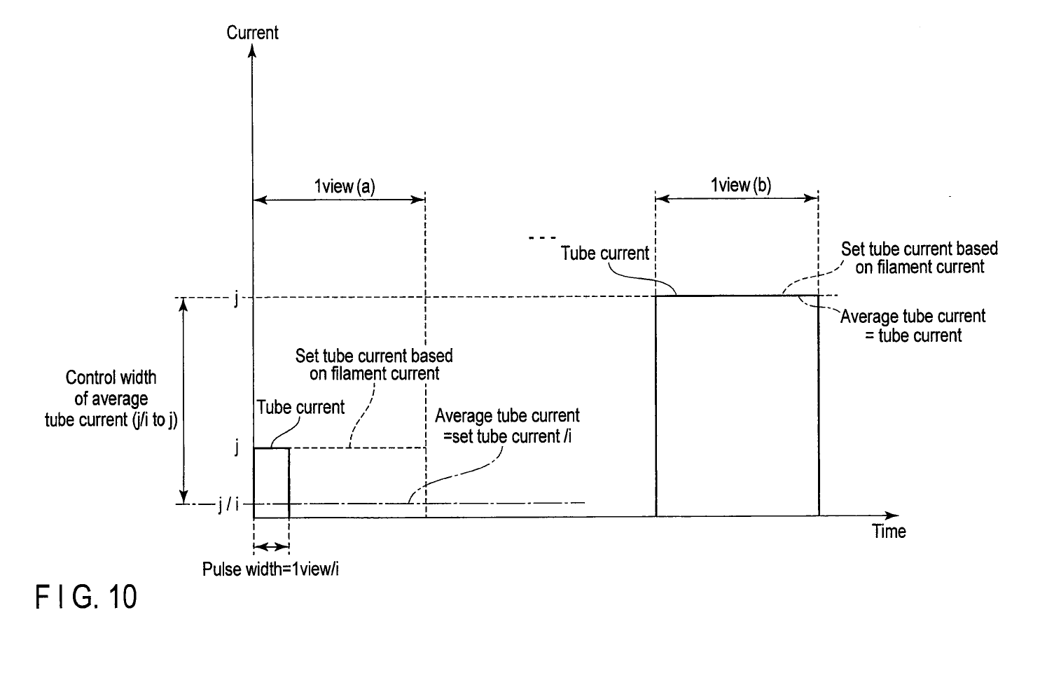

FIG. 10 is a graph for explaining the maximum and minimum values of a tube current. As shown in FIG. 10, the pulse width of a grid voltage is 1/i of a view interval. As shown in FIG. 10, j represents a tube current (the set tube current based on a filament current). The set tube current based on a filament current is a tube current corresponding to a value as a reference when the bias voltage of the grid is 0 or a given value. At this time, as indicated by view (a) in FIG. 10, the minimum tube current (average tube current) is 1/i times a set tube current, i.e., j/i. As indicated by view (b) in FIG. 10, the maximum tube current (average tube current) is one time the tube current, i.e., j. As shown in FIG. 10, the control width of a tube current (average tube current) is j/i to j.

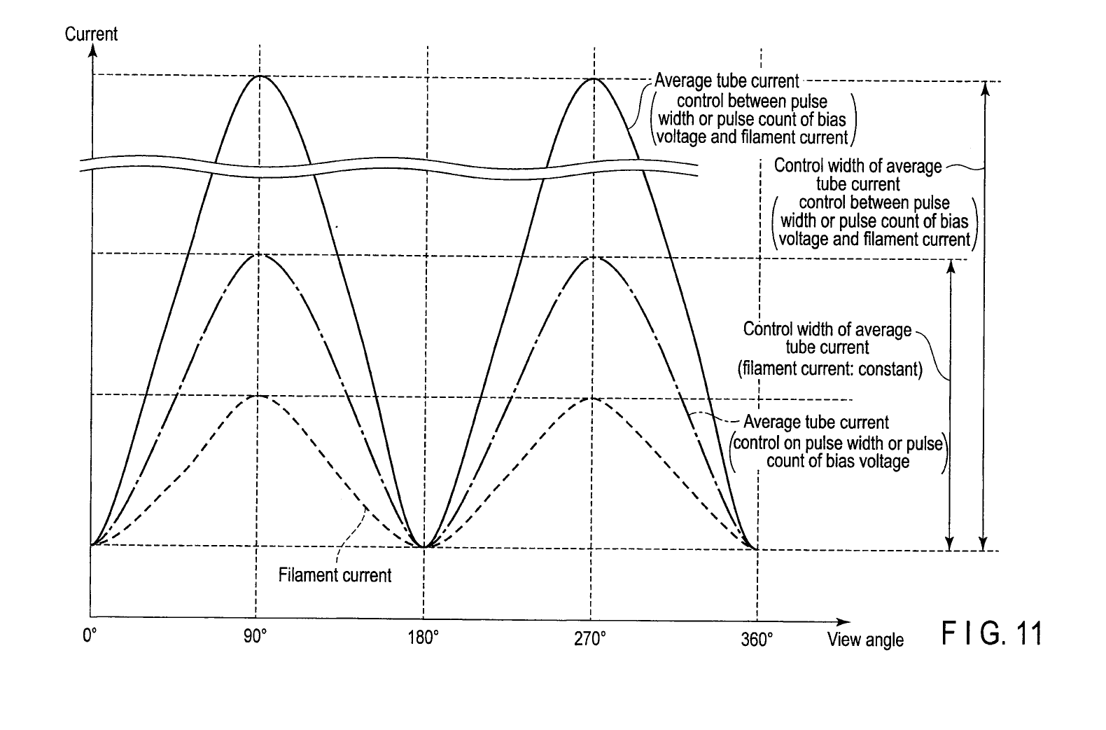

FIG. 11 is a graph showing an example of the control width of a tube current corresponding to a view angle. As shown in FIG. 11, this modification improves the control width of an average tube current as compared with the control width of an average tube current concerning a constant filament current.

The arrangement described above can obtain the following effects.

The X-ray CT apparatus according to this embodiment can control an X-ray tube current at high speed without changing a focus size by controlling at least one of the pulse count and pulse width of a grid voltage (bias voltage). That is, this X-ray CT apparatus can control an X-ray tube current at high speed without changing the resolution of projection data. This makes it possible to control an X-ray tube current at high speed and reduce radiation exposure on an object without causing resolution unevenness of the image reconstructed from projection data, a deterioration in image quality due to resolution unevenness, and artifacts.

In addition, the X-ray CT apparatus according to this modification indirectly controls a tube current by controlling the filament heating power supply 143 in accordance with the position of the X-ray tube 111 relative to an object. With this control, according to the modification, it is possible to further reduce radiation exposure on an object without degrading image quality. That is, according to the modification, it is possible to indirectly adjust the basic value of a tube current (a reference value for the bias voltage of the grid which is 0 or a given value) by adjusting a current (filament current) flowing in the filament in accordance with the estimated position of the X-ray tube 111.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An X-ray computed tomography apparatus comprising:
an X-ray tube including a cathode, an anode, and a grid placed between the cathode and the anode;
a bias power supply configured to generate a bias voltage applied to the grid to control a tube current between the cathode and the anode; and
an X-ray control unit configured to apply the bias voltage as a pulse string which generates a constant tube current and control at least one of a pulse count and a pulse width of the bias voltage for each predetermined period.

2. The X-ray computed tomography apparatus according to claim 1, further comprising a data acquisition unit configured to acquire projection data concerning an object based on an output from a X-ray detector,
wherein the X-ray control unit controls at least one of the pulse count and the pulse width of the bias voltage in synchronism with a data acquisition period of the data acquisition unit for the each data acquisition period.

3. The X-ray computed tomography apparatus according to claim 2, wherein the X-ray control unit controls at least one of the pulse count and the pulse width of the bias voltage for each view.

4. The X-ray computed tomography apparatus according to claim 1, further comprising a filament heating power supply configured to generate a filament current supplied to a filament forming the cathode,
wherein the X-ray control unit controls the filament heating power supply to change a magnitude of the tube current in accordance with a position of the X-ray tube relative to an object.

5. The X-ray computed tomography apparatus according to claim 4, wherein the X-ray control unit controls the filament heating power supply to change the tube current in accordance with a view direction of the X-ray tube relative to the object.

6. The X-ray computed tomography apparatus according to claim 4, wherein the X-ray control unit controls the filament heating power supply such that the tube current when the X-ray tube is located in a direction perpendicular to a top on which the object is placed becomes smaller than the tube current when the X-ray tube is located in a short-axis direction of the top.
